(19) United States
(12) Patent Application Publication
(10) Pub. No.: US 2007/0265583 A1
(43) Pub. Date: Nov. 15, 2007

(54) Catheter input device

(75) Inventor: Gary J. Secora, Menomonee Falls, WI (US)

Correspondence Address: PETER VOGEL;GE HEALTHCARE, 3000 N. GRANDVIEW BLVD., SN-477, WAUKESHA, WI, 53188, US

(73) Assignee: General Electric Company

(21) Appl. No.: 11/431,318

(22) Filed: May 10, 2006

(51) Int. Cl. A61M 25/00; A61M 39/02; A61M 5/00; A61M 25/16; A61M 25/18

(52) U.S. Cl. 604/264; 604/539

(57) ABSTRACT

A catheter input device for use with a medical instrument and a sensor device. The catheter input device includes an enclosure having a front portion. The catheter input device also includes a plurality of multi-contact receptacles mounted in the front portion. The catheter input device also includes a circuit board disposed in the enclosure and coupled to each multi-contact receptacle. Each multi-contact receptacle is configured to couple with the sensor device and the circuit board is configured to couple with the medical instrument.

### FIELD OF THE INVENTION

The present invention relates generally to the field of catheter input devices and specifically to a catheter input device for coupling a medical instrument to a catheter.

### BACKGROUND OF THE INVENTION

Typically, a catheter used in an invasive study will use intermediary cable to connect to the data acquisition device. This cable may be referred to as a catheter connector cable. The connector style on the data acquisition device end of the cable may include individual pins in large part because many current catheter input devices in the industry use a number of pin receptacles to bring in data from each of multiple catheters used on the patient. Upcoming imaging and data acquisition software may use a new connection style. This may prompt a change to a single circular connector (receptacle), on a catheter input device. New catheters or catheter cables may start to be produced with the circular plug style, however existing cables may go to waste if they are not usable.

Thus there is a need for a catheter input device that is able to directly couple to a number of circular plug styles from each catheter used on a patient. There is also a need for a catheter cable adapter that is able to couple a circular receptacle of a catheter input device with a catheter or catheter cable of an older pin configuration.

### SUMMARY OF THE INVENTION

One embodiment of the invention relates to a catheter input device for use with a medical instrument and a sensor device. The catheter input device includes an enclosure having a front portion. The catheter input device also includes a plurality of multi-contact receptacles mounted in the front portion. The catheter input device also includes a circuit board disposed in the enclosure and coupled to each multi-contact receptacle. Each multi-contact receptacle is configured to couple with the sensor device and the circuit board is configured to couple with the medical instrument.

Another embodiment of the invention relates to a system including a patient bed configured to support a patient in a patient care facility. The system also includes a medical imaging device configured to obtain internal medical images of the patient. The system also includes a catheter configured to be inserted into the patient to cooperate with the medical imaging device to obtain the medical images. The system also includes a catheter input device coupled to the medical imaging device and the catheter. The catheter input device includes an enclosure having a front portion. The catheter input device also includes a plurality of multi-contact receptacles mounted in the front portion. The catheter input device also includes a circuit board disposed in the enclosure and coupled to each multi-contact receptacle. Each multi-contact receptacle is configured to couple with the catheter and the circuit board is configured to couple with the medical imaging device.

Another embodiment of the invention relates to a catheter input device for use with a medical instrument and a sensor device. The catheter input device including an enclosure having a front portion. A plurality of multi-contact receptacles mounted in the front portion, with each receptacle having ten contacts arranged in a circular pattern. A circuit board disposed in the enclosure and coupled to each multi-contact receptacle, wherein each multi-contact receptacle is configured to couple with the sensor device and the circuit board is configured to couple with the medical instrument.

### BRIEF DESCRIPTION OF THE DRAWINGS

FIG. 1 is a perspective view of a system including a catheter input device according to one exemplary embodiment.

FIG. 2 is a perspective partial cut-away view of a catheter input device of FIG. 1 according to one exemplary embodiment.

FIG. 3 is a perspective view of a catheter cable adapter of FIG. 2 according to one exemplary embodiment.

### DETAILED DESCRIPTION OF THE PREFERRED EMBODIMENTS

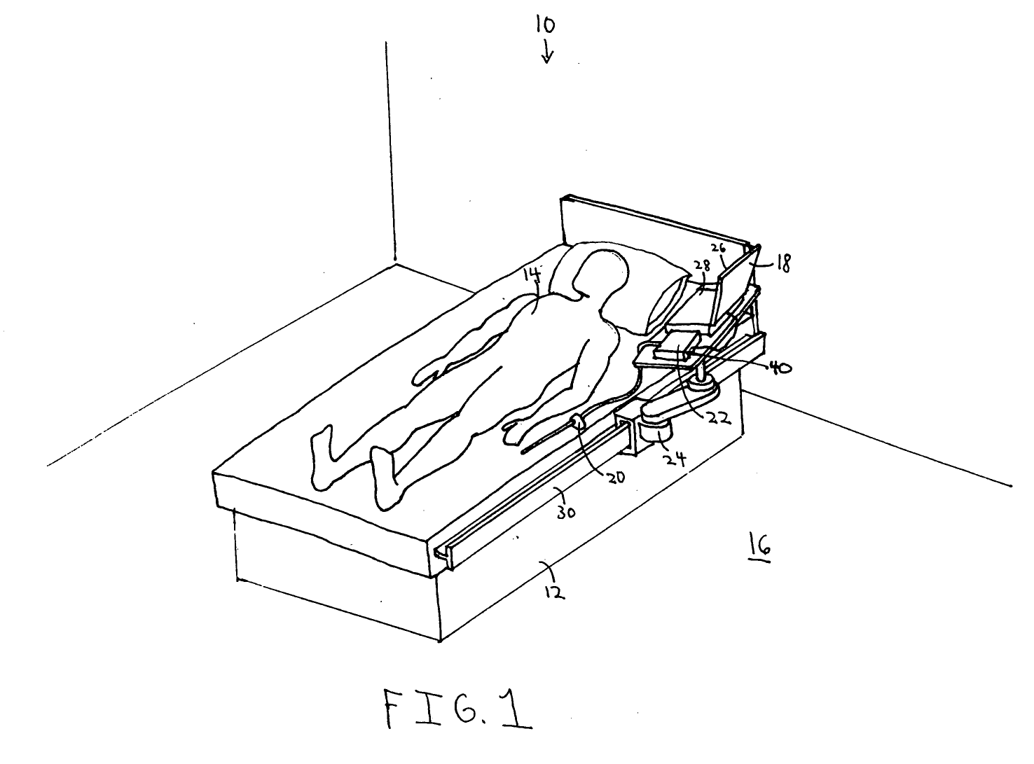

Referring to FIG. 1, a system 10 includes a patient bed 12, a patient care facility 16, a medical instrument 18 for example a medical imaging device, a sensor device 20 for example a catheter, and a catheter input device 22. Patient bed 12 is configured to support a patient 14 in patient care facility 16 so that medical imaging device 18 may obtain internal medical images of patient 14 using catheter 20. In various exemplary embodiments, patient bed 12 may be any suitable bed of past, present, or future design that is capable of supporting a patient. In one exemplary embodiments, patient 14 may be a human. In other exemplary embodiments, patient 14 may be another anatomical structure such as a dog, cat, horse, or primate. In another exemplary embodiment, patient care facility 16 may be a hospital or hospital room, while in still other exemplary embodiments, facility 16 may be any facility suitable for performing medical imaging on patient 14.

Medical imaging device 18 may include a mounting structure 24 and cooperates with sensor device or catheter 20 to obtain medical images from patient 14. Imaging device 18 may be of a laptop-style configuration with a flip-up screen 26 that displays imaging data or options and a horizontal work area 28. In various exemplary embodiments imaging device 18 may be any imaging device of past, present, or future design such as an ultrasound device, x-ray device, or thermal imaging device. In one example embodiment imaging device 18 may be configured to process the data retrieved using an imaging processor.

Mounting structure 24 is configured to support imaging device 18 and is coupled to bed 12 via a rail 30. In another exemplary embodiment, mounting structure 24 may be coupled to bed 12 via means other than a rail. Mounting structure 24 is capable of positioning imaging device 18 in a variety of locations as desired by a clinician for various reasons such as for best viewing or so as to not be obstructing any procedure performed on patient 14. In other exemplary embodiments, mounting structure may be a cart that is not directly coupled to bed 12.

Sensor device or catheter 20 is configured to obtain medical imaging data from the interior of patient 14. In various exemplary embodiments, catheter 20 may be of any past, present, or future design that allows for imaging data retrieval from the interior of patient 14. In another exemplary embodiment, sensor device 20 may be configured to take readings externally of patient 14.

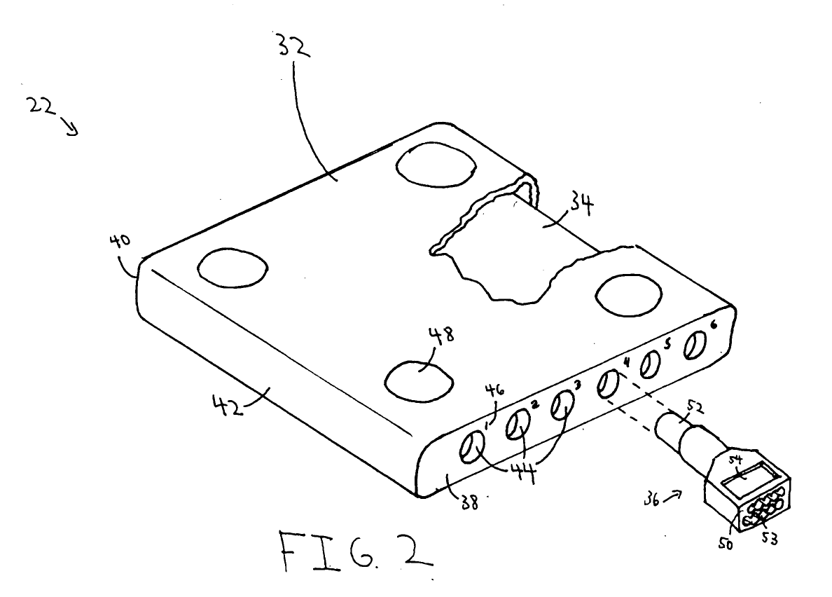

Referring to FIG. 2, catheter input device 22 is configured to couple imaging device 18 with catheter 20. Input device 22 generally includes an enclosure 32, a circuit board 34, and a catheter cable adapter 36. Enclosure 32 encloses and is intended to protect circuit board 34 from damage due to such things as jarring, moisture, and debris. Enclosure 32 includes a front portion 38, a rear portion 40, and body portion 42.

Front portion 38 defines a multi-contact receptacle 44, that is configured to coupled with sensor device 20. Each receptacle 44 is coupled to circuit board 34, thus coupling sensor device 20 to circuit board 34. Each receptacle 44 may be marked by an indicator 46 to aid in identification of each receptacle 44. Although the illustrated exemplary embodiment defines six receptacles 44, in other exemplary embodiments, more or fewer than six receptacles 44 may be used to couple to any number of sensor devices 20. In one exemplary embodiment receptacle 44 may be a Redel® receptacle configured to receive a Redel® plug or contact, while in other exemplary embodiments, other types of past, present, or future connection configurations may be used. The receptacle 44, in one embodiment, includes ten contacts arranged in a circular configuration. The contacts can be gold-plated. The receptacle can be composed of material that can be sterilized by gas or by steam, for example polysulfone. The receptacle 44 may also include a keying system and a locking system configured to engage a corresponding plug. Although the illustrated exemplary embodiment shows each receptacle 44 to be circular, in other exemplary embodiments, receptacles 44 may be of any other shape that facilitates connection of sensor device 20 to imaging device 18.

Rear portion 40 is configured to couple circuit board 34 to imaging device 18. In various exemplary embodiments, rear portion 40 may include either a removable or unitary cable capable of interfacing with imaging device 18 using a protocol such as serial, IEEE-1284, universal serial bus (USB) or IEEE-1394 communication or any other past, present, or future wired communication protocol. In other exemplary embodiments, rear portion 40 may facilitate wireless communication between circuit board 34 and imaging device 18 using any past, present, or future wireless communication protocol.

Body portion 42 may be configured to form a substantial seal with front portion 38 and rear portion 40 in order to protect circuit board 34. Body portion 42 includes a raised section 48 that mates with an indentation on a second catheter input device 22 so that they may be stacked or mounted on top of each other for coupling to imaging device 18 in the event that more connections are needed than provided by a single input device 22. In various exemplary embodiments, body portion may be composed of a rubber, a plastic, a metal, any other suitable material, or any combination thereof.

Circuit board 34 is generally configured to facilitate the connection between multiple sensor devices 20 with imaging device 18. Circuit board is coupled to each multi-contact receptacle, and thus each sensor device 20, and to imaging device 18. In one exemplary embodiment, circuit board 34 may selectively couple multiple sensor devices 20 to imaging device 18 and only allow selected sensor devices 20 to be electrically coupled to imaging device 18. In another exemplary embodiment, circuit board 34 may couple every sensor device 20 with imaging device 18 via multiplexing or using any other past, present, or future data communication method. In other exemplary embodiments, circuit board 34 may include additional circuitry and components appropriate for the medical procedure intended to be conducted.

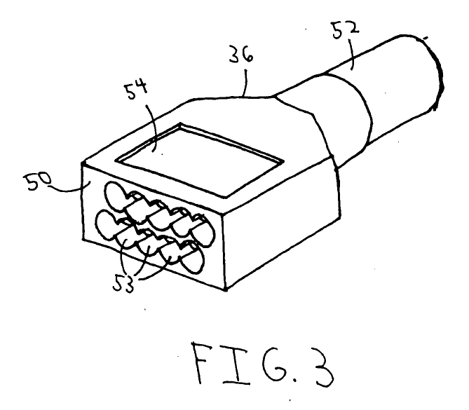

Referring to FIG. 3, catheter cable adapter 36 is configured to interface between circuit board 34 and sensor device 20 if the catheter cable coupled to sensor device 20 is of a connector design other than that of receptacle 44. Catheter cable adapter 36 includes a first end 50 and a second end 52. First end 50 is configured to receive a plurality of catheter or sensor device 20 input contacts from a pin-type catheter cable. First end 50 is illustrated as defining ten circular openings 53 that are arranged in a staggered configuration to conserve space. First end 50 also includes a display area 54 that may be used to indicate characteristics about a coupled device or a pin configuration. Second end 52 is configured to engage one multi-contact receptacle 44. First end 50 and second end 52 may be electrically and mechanically connected. Adapter 36 may be composed of a plastic, a rubber, a metal, any other suitable material, or any combination thereof.

It is noted that while in the illustrated exemplary embodiment a ten pin configuration is used for receiving contacts from sensor device 20, in other exemplary embodiments a different number of pins may be used, a different shape of pins may be used, or a different style that does not use pins may be used in order to connect sensor devices 20 using cable connector designs different than the design of receptacle 44.

Although specific shapes of each element have been set forth in the drawings, each element may be of any other shape that facilitates the function to be performed by that element. For example, receptacles 44 are shown to have a generally circular shape, however, in other exemplary embodiments receptacles 44 may be square shaped so long as coupling of sensor device 20 to circuit board 34 is facilitated.

Although imaging system 10 is illustrated as including multiple features utilized in conjunction with one another, system 10 may alternatively utilize less than all of the noted mechanisms or features. For example, in other exemplary embodiments, adapter 36 may be omitted with sensor device 20 coupling directly to receptacle 44 if the coupling cable is of the appropriate connection type.

For purposes of this disclosure, the term “coupled” means the joining of two components (electrical or mechanical) directly or indirectly to one another. Such joining may be stationary in nature or movable in nature. Such joining may be achieved with the two components (electrical or mechanical) and any additional intermediate members being integrally defined as a single unitary body with one another or with the two components or the two components and any additional member being attached to one another. Such joining may be permanent in nature or alternatively may be removable or releasable in nature

The present disclosure has been described with reference to example embodiments, however workers skilled in the art will recognize that changes may be made in form and detail without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example is manifestly intended to be as broad as possible. For example, unless specifically otherwise noted a single particular element may also encompass a plurality of such particular elements.

It is also important to note that the construction and arrangement of the elements of the system as shown in the preferred and other exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes and omissions may be made in the design, operating conditions and arrangement of the preferred and other exemplary embodiments without departing from the spirit of the present subject matter.

What is claimed is:

1. A catheter input device for use with a medical instrument and a sensor device, the catheter input device comprising:
an enclosure having a front portion;
a plurality of multi-contact receptacles mounted in the front portion; and
a circuit board disposed in the enclosure and coupled to each multi-contact receptacle, wherein each multi-contact receptacle is configured to couple with the sensor device and the circuit board is configured to couple with the medical instrument.

2. The catheter input device of claim 1, including a catheter cable adapter comprising:
a first end and a second end, with the first end configured to receive a plurality of catheter input contacts and the second end is configured to engage one of the multi-contact receptacles, wherein the first end and second end are electrically and mechanically connected.

3. The catheter input device of claim 2, wherein the catheter input contacts are arranged in a staggered configuration.

4. The catheter input device of claim 2, wherein each multi-contact receptacle includes ten contacts arranged in a circular pattern.

5. The catheter input device of claim 1, including another catheter input device mounted atop the other catheter input device and coupled to the medical instrument.

6. The catheter input device of claim 1, wherein at least six multi-contact receptacles are mounted in the front portion.

7. The catheter input device of claim 1, wherein the sensor device is an ultrasound imaging array and the medical instrument is an ultrasound imaging system.

8. A system comprising:
a patient bed configured to support a patient in a patient care facility;
a medical imaging device configured to obtain internal medical images of the patient;
a catheter configured to be inserted into the patient to cooperate with the medical imaging device to obtain the medical images; and
a catheter input device coupled to the medical imaging device and the catheter, the catheter input device comprising:
an enclosure having a front portion;
a plurality of multi-contact receptacles mounted in the front portion; and
a circuit board disposed in the enclosure and coupled to each multi-contact receptacle, wherein each multi-contact receptacle is configured to couple with the catheter and the circuit board is configured to couple with the medical imaging device.

9. The system of claim 8, including a catheter cable adapter comprising:
a first end and a second end, with the first end configured to receive a plurality of catheter input contacts and the second end is configured to engage one of the multi-contact receptacles, wherein the first end and second end are electrically and mechanically connected.

10. The system of claim 9, wherein the catheter input contacts are arranged in a staggered configuration.

11. The system of claim 10, wherein each multi-contact receptacle includes ten contacts arranged in a circular pattern.

12. The system of claim 8, including another catheter input device mounted atop the other catheter input device and coupled to the medical imaging device.

13. The system of claim 8, wherein at least 6 multi-contact receptacles are mounted in the front portion.

14. The system of claim 8, wherein the catheter includes an ultrasound imaging array and the medical imaging device is an ultrasound imaging system.

15. A catheter input device for use with a medical instrument and a sensor device, the catheter input device comprising:
an enclosure having a front portion;
a plurality of multi-contact receptacles mounted in the front portion, with each receptacle having ten contacts arranged in a circular pattern; and
a circuit board disposed in the enclosure and coupled to each multi-contact receptacle, wherein each multi-contact receptacle is configured to couple with the sensor device and the circuit board is configured to couple with the medical instrument.

16. The catheter input device of claim 15, including another catheter input device mounted atop the other catheter input device and coupled to the medical instrument.

17. The catheter input device of claim 15, wherein at least six multi-contact receptacles are mounted in the front portion.

18. The catheter input device of claim 15, wherein the sensor device is an ultrasound imaging array and the medical instrument is an ultrasound imaging system.

19. The catheter input device of claim 15, including a catheter cable adapter comprising:
a first end and a second end, with the first end configured to receive a plurality of catheter input contacts and the second end is configured to engage one of the multi-contact receptacles, wherein the first end and second end are electrically and mechanically connected.

20. The catheter input device of claim 19, wherein the catheter input contacts are arranged in a staggered configuration.
